(19) United States
(12) Patent Application Publication
(10) Pub. No.: US 2026/0207070 A1
(43) Pub. Date: Jul. 23, 2026

(54) NON-INVASIVE CORONARY SLOW FLOW MONITORING DEVICE AND METHOD

(71) Applicant: RheoDevice Technology Co., Ltd., Taipei City (TW)

(72) Inventor: CHIA-HSING LIU, Taipei City (TW)

(21) Appl. No.: 19/034,504

(22) Filed: Jan. 22, 2025

(51) Int. Cl. A61B 5/026; A61B 5/00; A61B 5/11; G16H 40/63; G16H 50/30

(52) U.S. Cl. CPC A61B 5/026; A61B 5/1102; A61B 5/7278; A61B 5/7282; G16H 40/63; G16H 50/30

(57) ABSTRACT

A non-invasive coronary slow flow monitoring device and a non-invasive coronary slow flow monitoring method. The device includes an acquisition member, an analysis member, and a marking member. The acquisition member acquires a continuous blood pressure waveform of a subject within a target period. The analysis member is communicatively connected to the acquisition member. The analysis member converts the continuous blood pressure waveform acquired by the acquisition member into a characteristic waveform having at least seven extreme values per cycle. The marking member communicatively connected to the analysis member determines whether or not a time interval between occurrences of the sixth extreme value and the seventh extreme value of each cycle of the characteristic waveform is greater than a preset threshold, and in response to the time interval being greater than the preset threshold, outputs a marked signal, or performs marking to form marked data.

### FIELD OF THE DISCLOSURE

The present disclosure relates to a non-invasive monitoring device and a non-invasive monitoring method, and more particularly to a non-invasive coronary slow flow monitoring device and a non-invasive coronary slow flow monitoring method.

### BACKGROUND OF THE DISCLOSURE

Coronary slow flow (CSF) is a clinically common (accounting for about 34% of coronary artery circulation abnormality cases) and critical coronary artery circulation abnormality. CSF is characterized by a significant slowing of blood flow in the distal coronary arteries during coronary angiography without apparent obstruction or stenosis of the coronary arteries. This phenomenon was first proposed by Tambe et al. in 1972 and is still a popular field of clinical research. Typical symptoms of coronary slow flow are similar to those of angina, in which patients may experience chest pain and chest tightness, especially after physical activity. Because the symptoms of CSF are similar to those of coronary obstructive heart disease, CSF is often misdiagnosed. For example, it is common for young people to have the symptoms of paroxysmal angina pectoris and other symptoms on a daily basis but the symptoms are not present when tests are performed at the hospital, resulting in misdiagnoses. In addition, the phenomenon of coronary slow flow may also lead to arrhythmia, myocardial ischemia, and even increase the risk of acute coronary syndrome (such as myocardial infarction).

However, the phenomenon of coronary slow flow does not show specific symptoms in every patient and specific testing methods are required for doctors to provide diagnosis. Existing ways for detecting coronary slow flow mainly include invasive and non-invasive tests. An invasive test such as coronary angiography is the gold standard for diagnosing CSF. The coronary angiography is a technique includes injecting radiocontrast agent into coronary arteries and observing the speed of blood flow in the coronary arteries through X-rays, and is usually combined with flow fractional flow reserve (FFR), which measures a pressure ratio within the coronary arteries to assess whether or not functional stenosis has occurred in the blood vessel. Techniques of intravascular ultrasound (IVUS) use ultrasound to detect an inner wall of the coronary arteries, and provide detailed images of the vascular structure to assist in detecting lesions in coronary artery walls. Further, optical coherence tomography (OCT) has higher resolution and can finely detect coronary arterial intima and microscopic lesions. Non-invasive techniques include cardiac magnetic resonance imaging (CMR), an imaging technique capable of assessing myocardial perfusion during resting and stress conditions and suitable for detecting cardiac blood flow and myocardial ischemia; positron emission tomography (PET) scanning is used to track radioactive markers to assess myocardial metabolism and blood flow, and is another precise tool for measuring flow reserve; coronary computed tomography angiography (CCTA) is a non-invasive three-dimensional imaging technology that can clearly display the anatomy of the coronary arteries and is often used to rule out coronary artery stenosis; echocardiography uses ultrasound to evaluate cardiac function and hemodynamics, so as to provide information on ventricular and atrial function and help rule out other structural heart diseases.

However, regardless of being invasive or non-invasive, the above existing technologies require a certain scale of medical institutions and more professional and expensive medical instruments to obtain the test data. Further, a continuous and daily monitoring cannot be carried out, and results may be limited by the status of the subject on the day of test.

### SUMMARY OF THE DISCLOSURE

In response to the above-referenced technical inadequacies, the present disclosure provides a non-invasive coronary slow flow monitoring device and a non-invasive coronary slow flow monitoring method.

In order to solve the above-mentioned problems, one of the technical aspects adopted by the present disclosure is to provide a non-invasive coronary slow flow monitoring device. The non-invasive coronary slow flow monitoring device includes an acquisition member, an analysis member, and a marking member. The acquisition member is used to acquire at least one continuous blood pressure waveform of a subject within at least one target period. The analysis member is communicatively connected to the acquisition member. The analysis member converts the continuous blood pressure waveform acquired by the acquisition member into a characteristic waveform having at least a first extreme value, a second extreme value, a third extreme value, a fourth extreme value, a fifth extreme value, a sixth extreme value, and a seventh extreme value per cycle. The marking member is communicatively connected to the analysis member. The marking member determines whether or not a time interval between occurrences of the sixth extreme value and the seventh extreme value of each cycle of the characteristic waveform is greater than a preset threshold. In response to the time interval being greater than the preset threshold, the marking member outputs a marked signal, or performs marking to form marked data.

In order to solve the above-mentioned problems, another one of the technical aspects adopted by the present disclosure is to provide a non-invasive coronary slow flow monitoring device. The non-invasive coronary slow flow monitoring device includes a control element, a memory element, and a communication element. The control element includes an acquisition member, an analysis member, and a marking member. The acquisition member is used to acquire at least one continuous blood pressure waveform of a subject within at least one target period. The analysis member is used to convert the continuous blood pressure waveform obtained by the acquisition member into a characteristic waveform having at least a first extreme value, a second extreme value, a third extreme value, a fourth extreme value, a fifth extreme value, a sixth extreme value, and a seventh extreme value per cycle. The marking member is used to determine whether or not a time interval between occurrences of the sixth extreme value and the seventh extreme value of each cycle of the characteristic waveform is greater than a preset threshold. In response to the time interval being greater than the preset threshold, the marking member performs marking to form marked data. The memory element is communicatively connected to the control element for storing the continuous blood pressure waveform or the marked data. The communication element is communicatively connected to the control element and the memory element. The communication element is used to externally receive the continuous blood pressure waveform or externally transmit the marked data.

In order to solve the above-mentioned problems, yet another one of the technical aspects adopted by the present disclosure is to provide a non-invasive coronary slow flow monitoring method. The non-invasive coronary slow flow monitoring method is run on a control device and includes the following processes. In an acquisition process, the control device obtains at least one continuous blood pressure waveform of a subject within at least one target period. In an analysis process, the control device converts the continuous blood pressure waveform into a characteristic waveform having at least a first extreme value, a second extreme value, a third extreme value, a fourth extreme value, a fifth extreme value, a sixth extreme value, and a seventh extreme value per cycle. In a marking process, the control device determines whether or not a time interval between occurrences of the sixth extreme value and the seventh extreme value of each cycle of the characteristic waveform is greater than a preset threshold, and in response to the time interval being greater than the preset threshold, the control device outputs a marked signal, or performs marking to form marked data.

One of the beneficial effects of the present disclosure is that, the non-invasive coronary slow flow monitoring device and the non-invasive coronary slow flow monitoring method provided by the present disclosure can mark possible abnormalities regarding the coronary slow flow phenomenon through continuous blood pressure waveforms. Therefore, specific subjects can be monitored and observed for a long time, and the overall structure of the device is simple and can be placed in a wearable device for daily use and monitoring of subjects. In addition, the cost of obtaining diagnostic data related to the coronary slow flow phenomenon can also be reduced.

That is, by using the non-invasive coronary slow flow monitoring device provided by the present disclosure, physiological data related to the coronary slow flow and having significance for making medical diagnosis can be obtained without the patient going to a medical institution of a certain scale and undergoing tests with more professional and expensive medical instruments. Furthermore, the subject can be monitored continuously and daily through the non-invasive coronary slow flow monitoring device disposed in the wearable device. Moreover, through the design of the marked data, comparatively more complete physiological data related to the coronary slow flow and having significance for making medical diagnosis can be formed by marking and classifying measurement data according to the condition and time of the subject during the test.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a functional block diagram of a non-invasive coronary slow flow monitoring device according to one embodiment of the present disclosure;

FIG. 2 is another functional block diagram of the non-invasive coronary slow flow monitoring device according to one embodiment of the present disclosure;

FIG. 3 is a schematic diagram of marked data according to one embodiment of the present disclosure;

FIG. 4 is a schematic diagram of a signal illustration of a continuous blood pressure waveform according to the present disclosure;

FIG. 5 is a schematic diagram of a characteristic waveform having at least seven extreme values per cycle in the present disclosure;

FIG. 6 is a schematic diagram illustrating a characteristic waveform according to one embodiment of the present disclosure;

FIG. 7 is another schematic diagram illustrating the characteristic waveform according to one embodiment of the present disclosure;

FIG. 8 is yet another schematic diagram illustrating the characteristic waveform according to one embodiment of the present disclosure;

FIG. 9 is still another schematic diagram illustrating the characteristic waveform according to one embodiment of the present disclosure; and

FIG. 10 is a flowchart of a non-invasive coronary slow flow monitoring method according to one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

Coronary slow flow is characterized by a significant slowing of blood flow in the distal coronary arteries during coronary angiography without obvious obstruction or stenosis of the coronary arteries. The pathological basis of coronary slow flow may be related to microvascular dysfunction and endothelial cell dysfunction. These abnormalities may lead to slowed blood flow or even ischemia. According to researches, in a specific characteristic waveform related to the arterial blood pressure wave (i.e., a characteristic waveform having at least seven extreme values described hereinafter), a time interval between two extreme values can effectively reflect physiological phenomena correlating to microvascular dysfunction, such that physiological data with diagnostic significance and related to coronary slow flow can be formed. Accordingly, the present disclosure provides a non-invasive coronary slow flow monitoring device that can efficiently provide physiological data with diagnostic significance and related to coronary slow flow.

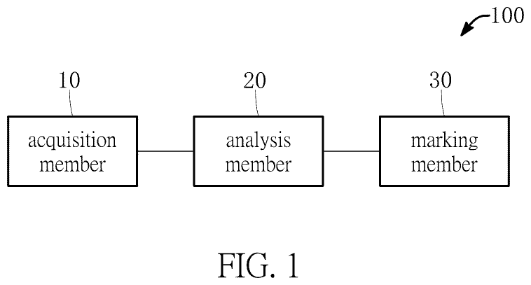

Referring to FIG. 1, the present disclosure provides a non-invasive coronary slow flow monitoring device 100, which includes an acquisition member 10, an analysis member 20, and a marking member 30. The acquisition member 10 is used to acquire at least one continuous blood pressure waveform of a subject within at least one target period. The analysis member 20 is communicatively connected to the acquisition member 10. The analysis member 20 converts the continuous blood pressure waveform acquired by the acquisition member 10 into a characteristic waveform having at least a first extreme value, a second extreme value, a third extreme value, a fourth extreme value, a fifth extreme value, a sixth extreme value, and a seventh extreme value per cycle. The marking member 30 is communicatively connected to the analysis member 20. The marking member 30 determines whether or not a time interval between occurrences of the sixth extreme value and the seventh extreme value of each cycle of the characteristic waveform is greater than a preset threshold, and in response to the time interval being greater than the preset threshold, the marking member 30 outputs a marked signal, or performs marking to form marked data.

The acquisition member 10 is used to obtain at least one continuous blood pressure waveform of a subject during at least one target period. The continuous blood pressure waveform can reflect blood pressure changes of the subject during the target period.

The continuous blood pressure waveform can be a waveform that describes the arterial blood pressure wave. The arterial blood pressure wave includes a percussion wave, a tidal wave, and a dicrotic wave, and is a pressure wave that propagates in the arteries during each of the contractions of the heart. The arterial blood pressure wave reflects the blood flow and the vascular response of the heart during a heartbeat cycle. The percussion wave is the primary pulse wave and represents the pressure wave produced by the heart when the heart pumps blood into the arteries during systole. The percussion wave is the main part of the overall blood pressure wave and corresponds to the peak of blood flow when the heart contracts. The tidal wave is a secondary wave that occurs after the heart contracts and enters diastole, and represents the process of blood returning from the artery and reflects the waveform characteristics of the blood flow as the blood flow gradually slows down. The dicrotic wave occurs when the aortic valve closes and is formed by the rebound of blood flow. The dicrotic wave is a characteristic of diastole, and is usually formed during the decline of the arterial blood pressure wave.

The continuous blood pressure waveform can also be a velocity waveform (a first-order derivative waveform) or an acceleration waveform (a second-order derivative waveform) of the arterial blood pressure wave.

The target period is defined as a specific time range of the continuous blood pressure waveform that encompasses multiple complete heartbeat cycles. Specifically, the target period is defined by a clear period start point and a period end point, and at least one or more complete cardiac cycles are covered between the two points in time. Each of the heartbeat cycles includes a systole and a diastole. The period start point and the period end point of the target period correspond to a specific set of time markers used to accurately calibrate the time range of the period for accurate analysis of the continuous blood pressure waveform.

The acquisition member 10 is implemented via a computer circuit and is designed to acquire at least one continuous blood pressure waveform from the subject within at least one target period, and the at least one continuous blood pressure waveform is such as an electropulsography (EPG). The acquisition member 10 can be implemented through a variety of data acquisition manners, including but not limited to sensors, data collection modules, or external data input devices. The acquisition member has automated data collection capabilities and can monitor the physiological state of the subject according to predefined conditions and time periods, and obtain continuous and complete blood pressure waveform data within the specified target period. The data can be stored immediately or delayed after recording for subsequent analysis and processing.

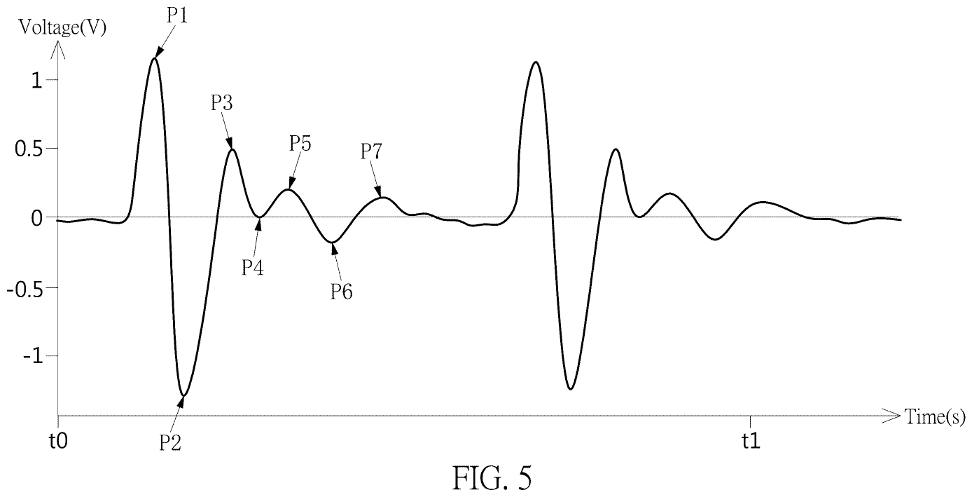

Referring to FIG. 1 and FIG. 5, the analysis member 20 is used to receive continuous blood pressure waveform data from the acquisition member 10, and convert the data into a second-order derivative waveform (i.e., an acceleration waveform) of the arterial blood pressure wave as a characteristic waveform. As shown in FIG. 5, the characteristic waveform has a first extreme value P1, a second extreme value P2, a third extreme value P3, a fourth extreme value P4, a fifth extreme value P5, a sixth extreme value P6, and a seventh extreme value P7 within a heartbeat cycle. The extreme values P1 to P7 include positive extreme values and negative extreme values, respectively. Here, a period between the first extreme value P1 and the fifth extreme value P5 corresponds to the systole of the arterial blood pressure wave, and a period between the fifth extreme value P5 and a next first extreme value P1 of the next cycle corresponds to the diastole of the arterial blood pressure wave. An extreme value refers to a local maximum value or a local minimum value reached by the waveform at a specific point in time. The positive extreme value represents the highest point reached upward by the waveform, and the negative extreme value is the lowest point reached downward by the waveform. In this waveform, the distribution of the extreme values reflects the changes in blood pressure over time, and the present disclosure puts special emphasis on the time interval between each of the extreme values.

The analysis member 20 can process the continuous blood pressure waveform obtained by the acquisition member 10, and can output a characteristic waveform having the first to seventh extreme values P1 to P7 in one cycle. The continuous blood pressure waveform can be in various forms, including waveforms that can reflect the original arterial blood pressure wave, a waveform that can reflect the first-order derivative of the original arterial blood pressure wave (the velocity waveform), or a waveform that can reflect the second-order derivative of the original arterial blood pressure wave (the acceleration waveform). For example, in one embodiment, regardless of the form of the input data, the analysis member 20 can implement dynamic identification through an internal algorithm and perform corresponding processing according to the type of the input continuous blood pressure waveform. When an original arterial blood pressure waveform is input, the analysis member 20 will perform two derivative operations on the original arterial blood pressure waveform to extract a second-order derivative waveform; if a first-order derivative waveform is input, the analysis member 20 will perform one derivative operation to generate the required second-order derivative waveform; when a second-order derivative waveform is input, the analysis member 20 will directly process and output the waveform. In order to realize this function, the analysis member 20 includes a digital signal processing module and corresponding software algorithms. The digital signal processing module includes a dynamic input analysis module used to automatically detect the type of input data and adapt to the corresponding processing logic. According to different input conditions, different derivative operation algorithms are used. When an original pressure waveform is input, a derivative operation module will perform two derivative operations, and when a first-order derivative is input, the system will only perform one derivative operation. After the data undergoes derivative operation, the data will be further processed by the digital filter module to filter out possible noise and interference signals to ensure that an output waveform remains smooth and has accurate characteristics. The implementation steps include first receiving data from the acquisition member 10 through a communication connection, then automatically detecting the type of data that is input, and subsequently executing an appropriate derivative algorithm for data processing. After completing the data processing, the analysis member 20 will transmit the result, that is, the second-order derivative waveform of the arterial blood pressure, to other parts of the system, such as the marking member 30 or a memory element 4 as described below. However, the above-mentioned is only one embodiment, and the present disclosure is not limited thereto. For example, the analysis member 20 may be not having the function of dynamic identification for input data; in one embodiment, when the continuous blood pressure waveforms output by the acquisition member 10 corresponding to the analysis member 20 are known, the analysis member 20 can also only have the function of first-order derivative, second-order derivative, or direct transmission.

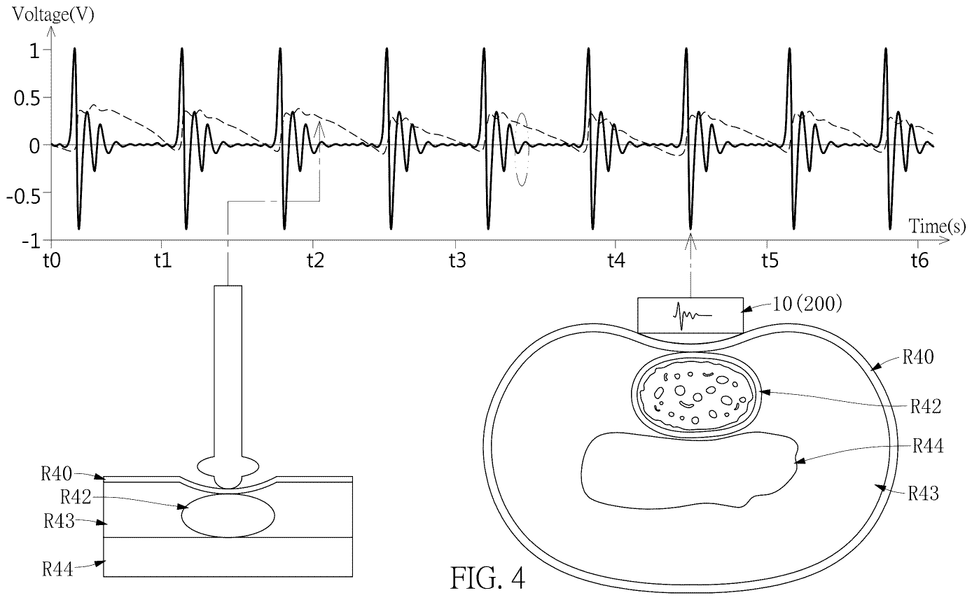

Referring to FIG. 4, FIG. 4 is a schematic diagram of a signal illustration of a continuous blood pressure waveform according to the present disclosure. The graph to the left side of FIG. 4 is, for example, a schematic diagram of a tonometer sampling the arterial blood pressure wave of a radial artery R42 of the subject, and the waveform of the arterial blood pressure wave that is formed is shown by the broken line. The graph to the right side of FIG. 4 is, for example, an acceleration sensor measuring a continuous blood pressure waveform (represented by a solid line) by directly contacting a skin R40 outside the radial artery R42 of the subject. The continuous blood pressure waveform measured by the acceleration sensor can be directly used as the characteristic waveform, and is the second-order derivative form of the waveform of the arterial blood pressure wave. In the figure, R43 is a tissue and R44 is a bone, which has the function of conducting heart pulse.

The marking member 30 is communicatively connected to the analysis member 20 and can output marked signals based on the characteristic waveforms from the analysis member 20 or perform marking to form marked data. The marking member 30 can determine the time interval between the sixth extreme value P6 and the seventh extreme value P7 in each cycle, and determine whether or not the time interval is greater than the preset threshold according to the preset threshold. In response to the time interval being greater than the preset threshold, the marking member 30 outputs the marked signal or performs marking to form the marked data.

The marking member 30 may include a determination logic module, a threshold comparison module, and a data recording module. The determination logic module analyzes the characteristic waveform received from the analysis member 20, determines occurrence times of the sixth extreme value P6 and the seventh extreme value P7 in each cycle, and calculates the time interval between the two extreme values. After the time interval between the sixth extreme value P6 and the seventh extreme value P7 is calculated, the threshold comparison module compares the time interval with the preset threshold, and, in response to the time interval being greater than the preset threshold, triggers the marked signal to be output. Once the marked signal is triggered, the data recording module can mark and record relevant marked information (such as a subject number, a data reception date and time, a data analysis period, a marking time, a time interval, and an accumulated marking number in the data analysis period, or a combination thereof) to form marked data.

The marking member 30 may perform the following processes for marking. In a data receiving process, the marking member 30 first receives the processed characteristic waveform data from the analysis member 20. In an extreme value determination process, the determination logic module inside the marking member 30 automatically identifies the sixth extreme value P6 and the seventh extreme value P7 in each cycle, and records the occurrence time of the sixth extreme value P6 and the seventh extreme value P7 (that are marked with timestamps). In a time interval calculation and comparison process, the marking member 30 calculates the time interval according to the timestamps of the sixth extreme value P6 and the seventh extreme value P7, and compares the time interval with the preset threshold. In response to the calculated time interval being greater than the preset threshold, the marked signal is triggered to be output. In a data recording and output process, in response to the calculated time interval being greater than the preset threshold, the marking member 30 forms marked data, and stores or outputs the marked data.

The preset threshold can be from 70 ms to 120 ms according to requirements. For example, the preset threshold can be 70 ms, 80 ms, 90 ms, 100 ms, 110 ms, and 120 ms, and the preset threshold is preferably 110 ms.

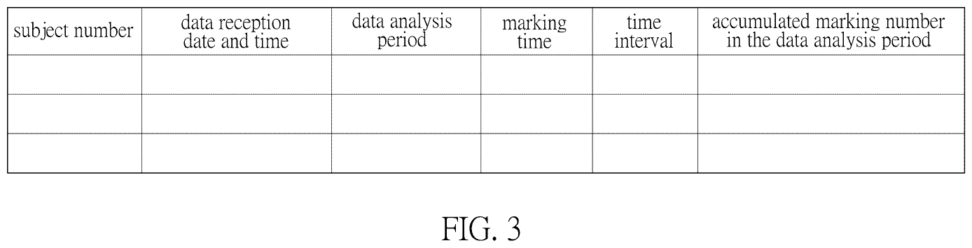

Referring to FIG. 3, FIG. 3 is a schematic diagram of marked data according to one embodiment of the present disclosure. The marked data consists of multiple fields, and each of the fields records data related to a blood pressure waveform analysis and marking of the subject during the target period. The marked data is stored in table form and contains the following fields. A filed of the subject number is a unique identification code used to distinguish different subjects; a corresponding number in the system (such as ID001) is assigned to each of the subjects in order to track blood pressure waveform data and marking results of the subject; the subject number is usually automatically generated or can be input by an operator. A field of the data reception date and time records a timestamp of a specific date and time when the system receives the raw data from the acquisition member 10 to ensure time synchronization and traceability of the data; the data reception date and time is usually in the format of: YYYY-MM-DD HH:MM:SS (for example, 2024-09-19 14:35:00), thereby ensuring accuracy to the second. A field of data analysis period specifies a specific time period (i.e., a target period) for analyzing data; that is, a range in which the system executes waveform analysis and marking operations is marked; the format of the data analysis period usually consists a start time and an end time, such as from YYYY-MM-DD HH:MM:SS to YYYY-MM-DD HH:MM:SS, indicating the specific duration of analysis (for example, from 2024-09-19 14:00:00 to 2024-09-19 14:30:00). A field of marking time records a specific point in time when marking occurs in the system, indicating the moment of marking when the time interval between the sixth extreme value P6 and the seventh extreme value P7 is greater than the preset threshold; the format of the marking time is also YYYY-MM-DD HH:MM:SS, so as to ensure the accuracy of the marking time and facilitate subsequent inspection. A field of the time interval displays an actual time interval between the sixth extreme value P6 and the seventh extreme value P7, and the unit of the time interval is milliseconds (for example, 120 ms). A field of the accumulated marking number in the data analysis period counts a number of times the marked signal is triggered during the entire data analysis period or a specific target period, and is used to display a frequency of abnormal situations within an analysis stage; this field can assist in evaluating an overall health status or blood pressure waveform abnormalities of the subject in a specific period; the recording format of this filed can be numeral, for example, 3 (times).

However, the above marked data format is only one of feasible embodiments, and the present disclosure is not limited thereto. In practice, specific fields for marked data can be flexibly adjusted and designed according to the requirements of the system. Different application scenarios may require adding or deleting fields. For example, more detailed physiological data records or additional health information of the subject may be added. Field configurations should be determined based on practical requirements to ensure that the system can effectively capture and store corresponding marked information and provide sufficient data for subsequent analysis. Therefore, as long as the fields include the time or number of times the marking member 30 performs marking, the form and content of other marked data can be flexible and allow customization according to different applications.

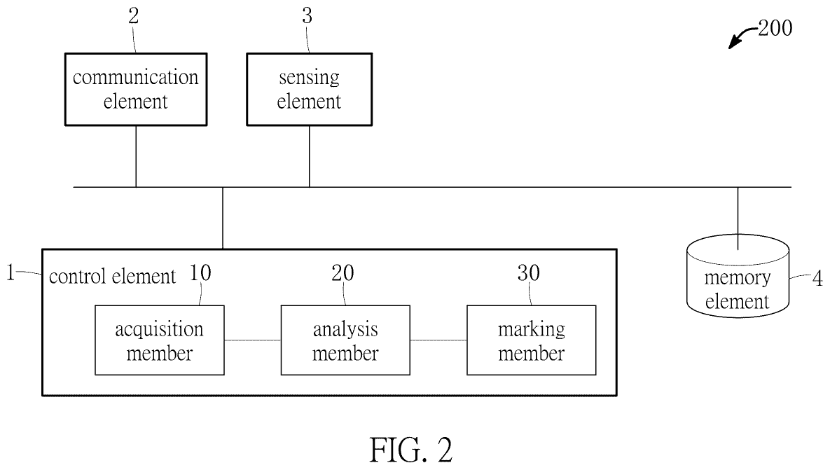

Referring to FIG. 2, FIG. 2 is the functional block diagram of a non-invasive coronary slow flow monitoring device 200 according to one embodiment of the present disclosure. The non-invasive coronary slow flow monitoring device 200 includes a control element 1, a memory element 4, and a communication element 2. The control element 1 includes an acquisition member 10, an analysis member 20, and a marking member 30. The acquisition member 10 is used to acquire at least one continuous blood pressure waveform of a subject within at least one target period. The analysis member 20 is used to convert the continuous blood pressure waveform obtained by the acquisition member 10 into a characteristic waveform having at least seven extreme values per cycle. The marking member 30 is used to determine whether or not a time interval between occurrences of the sixth extreme value P6 and the seventh extreme value P7 of each cycle of the characteristic waveform is greater than a preset threshold, and in response to the time interval being greater than the preset threshold, the marking member 30 performs marking to form marked data. The memory element 4 is communicatively connected to the control element 1 for storing the continuous blood pressure waveform or the marked data. The communication element 2 is communicatively connected to the control element 1 and the memory element 4 to externally receive the continuous blood pressure waveform or externally transmit the marked data.

Selectively, the non-invasive coronary slow flow monitoring device 200 may further include a sensing element 3 for sensing the subject to obtain the continuous blood pressure waveform.

The non-invasive coronary slow flow monitoring device 200 is an integrated device used to analyze the continuous blood pressure waveform of a subject, and to perform data acquisition, analysis, and marking during a detection process. The core operating component of the non-invasive coronary slow flow monitoring device 200 is the control element 1. The control element 1 includes multiple functional modules such as the acquisition member 10, the analysis member 20, and the marking member 30. The basic functions and structures of the acquisition member 10, the analysis member 20, and the marking member 30 are as described in the previous embodiments, and are coordinated by the control element 1 to be used in conjunction with the communication element 2, the sensing element 3 (optional), and the memory element 4 to complete the entire detection process.

The control element 1 is the central operating component of the non-invasive coronary slow flow monitoring device 200 and is responsible for the management and control of the entire system. The control element 1 is communicatively connected to the communication element 2, the sensing element 3 (optional), and the memory element 4, and executes a specific detection process according to externally or internally triggered commands. Specifically, the control element 1 will first obtain the continuous blood pressure waveform of the subject through the acquisition member 10 according to a preset process (such as a non-invasive coronary slow flow monitoring method described below), then the analysis member 20 analyzes the blood pressure waveform to form a characteristic waveform, and finally the marking member 30 determines the characteristic waveform to mark abnormal conditions. The control element 1 can be such as a processor, a controller, a microprocessor, a central processing unit, a signal processor, an embedded control module, a programmable logic controller, an industrial control computer, a system on a single chip, a field programmable gate array, and a control circuit.

The communication element 2 is used to exchange data with external devices to ensure that the non-invasive coronary slow flow monitoring device 200 can receive data from near-end or remote external devices or transmit analysis and marking results to external systems. The communication element 2 supports multiple communication protocols such as Wi-Fi®, Bluetooth®, or wired communication, and can be flexibly used in different medical environments.

The sensing element 3 is an optional module in the non-invasive coronary slow flow monitoring device 200. The non-invasive coronary slow flow monitoring device 200 needs to directly sense the subject through the sensing element 3 to obtain the continuous blood pressure waveform. The sensing element 3 may be a pressure wave sensor, a velocity wave sensor, or an acceleration wave sensor, and is used to detect the waveform of a radial artery or other partial arteries of the subject that can reflect the original arterial blood pressure wave, a waveform that can reflect the first-order derivative of the original arterial blood pressure wave (velocity waveform), or a waveform that can reflect the second-order derivative of the original arterial blood pressure wave (acceleration waveform). Specifically, the sensing element 3 may include a variety of sensing technologies, such as pressure sensors including piezoelectric sensors and strain gauges sensors, in which the piezoelectric sensors use the voltage change of the piezoelectric material to detect the pressure wave, while the strain gauges use the resistance change caused by the deformation of materials to record pulse waveform; photoplethysmography sensors (PPG sensors) are based on optical principles and use changes in blood volume to reflect the pulse waveform, and are often used in non-invasive pulse detection, such as finger or earlobe measurement; micro-pressure sensors are based on micro-electromechanical systems (MEMS) technology and due to having small size and high sensitivity, are suitable for portable or wearable apparatuses; pressure transducers convert arterial pressure changes into electrical signals, and are suitable for high-precision monitoring of pulse waveforms in medical environments; and accelerometers can directly measure the waveform (acceleration waveform) reflecting the second-order derivative of the original arterial blood pressure wave. However, the above examples are all embodiments of the present disclosure, and as long as the sensing element 3 can sense the waveform of the radial artery or other partial arteries of the subject that can reflect the original arterial blood pressure wave, the waveform (the velocity waveform) that can reflect the first-order derivative of the original arterial blood pressure wave, or the waveform (the acceleration waveform) that can reflect the second-order derivative of the original arterial blood pressure wave, the sensing element 3 of the present disclosure is not limited to the above-mentioned components.

The memory element 4 is a module used to store all data generated during the operation of the non-invasive coronary slow flow monitoring device 200. The data includes the continuous blood pressure waveform collected from the acquisition member 10, the characteristic data processed by the analysis member 20, and the marked data generated by the marking member 30. The data can be used by the control element 1 for retrospective analysis, and can also be used by medical personnel for subsequent diagnosis. The memory element 4 can also perform long-term data storage according to requirements and support the export of the data in different data formats.

It should be noted that the marked data is presented to interpreters (such as doctors, testing personnel, or researchers) in a way that can be adjusted according to requirements of interpretation. For example, the way can be based on the content of the marked data or based on the simplified results of the marked data. For example, texts such as “marked 5 times,” “abnormality detected,” or “abnormality not detected” can be displayed on a display based on the marked data. In addition, buzzers, flashes, etc. can also be used to indicate that the marked signal is output.

Accordingly, the non-invasive coronary slow flow monitoring device 200 is a multi-module cooperation system. Through the unified management of the control element 1, the acquisition member 10, the analysis member 20, and the marking member 30 can flexibly cooperate with each other to perform accurate physiological data analysis and mark abnormalities. Through the cooperative operation of the communication element 2, the sensing element 3 (optional), and the memory element 4, the device not only can receive and analyze external data in real-time, but also can store or share the results with other medical instruments. This enables the non-invasive coronary slow flow monitoring device 200 to be used in a variety of medical settings and provides comprehensive blood pressure waveform analysis and diagnostic support.

In addition, since the non-invasive coronary slow flow monitoring device 200 is mainly provided for data processing, the hardware structure of the device is relatively simple and can be integrated into existing electronic devices such as mobile phones, electronic bracelets, electronic glasses, computers, or other medical measurement devices. When made into a single device, the device can be made to be thin, lightweight, and miniaturized. Furthermore, if the non-invasive coronary slow flow monitoring device 200 includes the sensing element 3, the device can be made into a wearable device, such as a health bracelet, a head-mounted device, glasses, nursing clothes, etc., as long as a sensing part that is the sensing element 3 is in contact with the subject to measure and obtain the continuous blood pressure waveform of the subject, the wearable device can be various forms of wearable devices.

As mentioned above, by using the non-invasive coronary slow flow monitoring devices 100 and 200, there is no need to go to a medical institution of a certain size and perform measurements with more professional and expensive medical instruments in order to obtain physiological data with diagnostic significance and related to coronary slow flow. In addition, the subject can perform continuous and daily monitoring through the wearable non-invasive coronary slow flow monitoring device 200. Moreover, through the design of the marked data, the data can be marked and classified according to the status and time of the subject at the time of test, thereby forming relatively more complete physiological data with diagnostic significance and related to coronary slow flow.

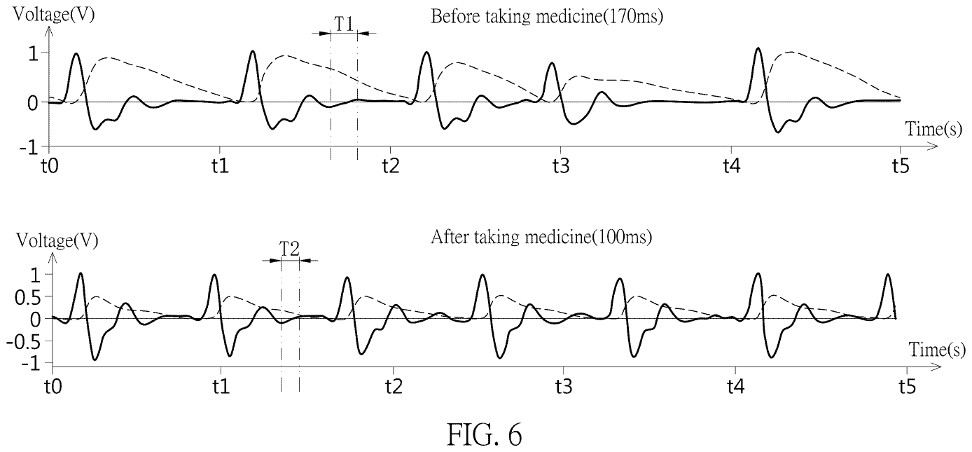

Hereinafter, an example of acquisition of physiological data with diagnostic significance and related to coronary slow flow through the non-invasive coronary slow flow monitoring devices 100 and 200 will be described with reference to FIG. 6 to FIG. 9. It should be noted that although the non-invasive coronary slow flow monitoring devices 100 and 200 of the present disclosure can also output characteristic waveforms and display the waveforms on an external display screen for review by interpreters (such as doctors or testing personnel), the non-invasive coronary slow flow monitoring devices 100 and 200 of the present disclosure mainly output marked data. However, for convenience herein, diagnostic significances that may be included in the characteristic waveforms are directly presented in FIG. 6 to FIG. 9 to present in a more intuitive way that the time interval between the sixth extreme value P6 and the seventh extreme value P7 of at least one cycle in the characteristic waveform is greater than the preset threshold (110 ms in this embodiment), thereby illustrating the special advantages of the non-invasive coronary slow flow monitoring devices 100 and 200 of the present disclosure in the medical field.

As shown in FIG. 6, FIG. 6 is a schematic diagram illustrating a characteristic waveform according to one embodiment of the present disclosure. Here, FIG. 6 shows two characteristic waveforms measured before and after taking arrhythmia-related medicines by a patient diagnosed with arrhythmia and instructed to take arrhythmia-related medicines. It can be seen from the characteristic waveform before taking the medicine that, during the target period of the patient before taking the medicine, a time interval T1 between the sixth extreme value and the seventh extreme value of the second heartbeat cycle is at most 170 ms, and 170 ms is already greater than the preset threshold that is set to be 110 ms. Therefore, when the heartbeat cycle is processed, the marking member 30 of the non-invasive coronary slow flow monitoring device 100 and 200 of the present disclosure will be triggered to output a marked signal, or the marking member 30 will be triggered to perform marking to form marked information, such that the marked information at least includes an accumulated marking number of 1. For external devices, for example, when the second heartbeat cycle is processed, the marked signal can be used to trigger a light-emitting device or a buzzer, or the status can be expressed as “1 time” or “abnormalities detected” on displays or reports. In addition, after detecting at least one target cycle, outputs that indicate result such as “accumulated N times” or “abnormalities detected” can be shown on the displays or reports (N is the cumulative number of times marked in the entire target cycle, and is a positive integer). On the other hand, the characteristic waveform of the patient having arrhythmia that is corrected after the patient takes the medicine shows that, during the target period, the time interval between the sixth extreme value and the seventh extreme value of each cycle is not greater than the preset threshold of 110 ms (a maximum time interval T2, for example, is 100 ms). In this case, the marking member 30 of the non-invasive coronary slow flow monitoring device 100, 200 of the present disclosure will not be triggered to output a marked signal, and no marked information will be formed. This status can be expressed externally as “0 times” or “no abnormality detected.” Accordingly, through such detection, whether or not patients with conditions related to coronary slow flow have improved after taking the medicine can be determined.

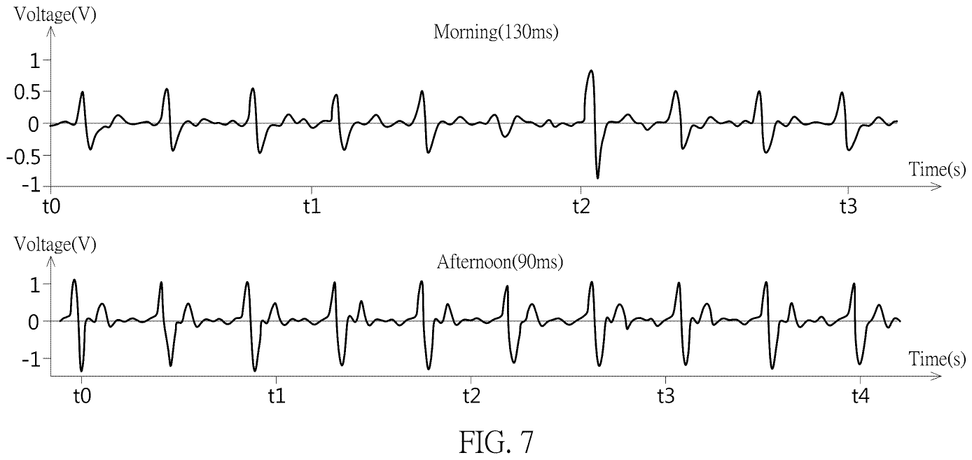

Next, referring to FIG. 7, FIG. 7 is shows characteristic waveforms of patients with morning hypertension in the morning and afternoon. It can be seen from the results of each of the characteristic waveforms in FIG. 7 that, the characteristic waveform analysis result in the morning is that the time interval between the sixth extreme value and the seventh extreme value of a certain heartbeat cycle is detected to be 130 ms, exceeding the preset threshold (110 ms) , while the characteristic waveform analysis results in the afternoon do not show that the time interval between the sixth extreme value and the seventh extreme value exceeds the preset threshold of 110 ms, for example, the maximum is 90 ms. In this way, by detecting marked signals or marked data in different periods of time, it is also possible to effectively understand the distribution of periods of time in which subjects may have abnormalities related to coronary slow flow. For this type of abnormal situation that may be triggered by the period of time that the subject is in or the time of routine activities (such as routine exercise, eating, sleeping, etc.), long-term monitoring is needed to provide more complete information having diagnostic significance and related to coronary slow flow to the interpreter. For example, the target period is set to be 24 hours to obtain marked signals or marked data for the 24 hours, such that the interpreter can understand the period of time and frequency of abnormalities of the subject. In addition, tracking of diseases triggered by the period of time that the diagnosed subject is in or the time of routine activities (such as routine exercise, eating, sleeping, etc.) can also be achieved by setting an interval target period. For example, a fixed time length is automatically measured at a fixed time in the morning and afternoon every day as the target period to generate marked signals or marked data, which can help interpreters to observe morning hypertension with more accurate information.

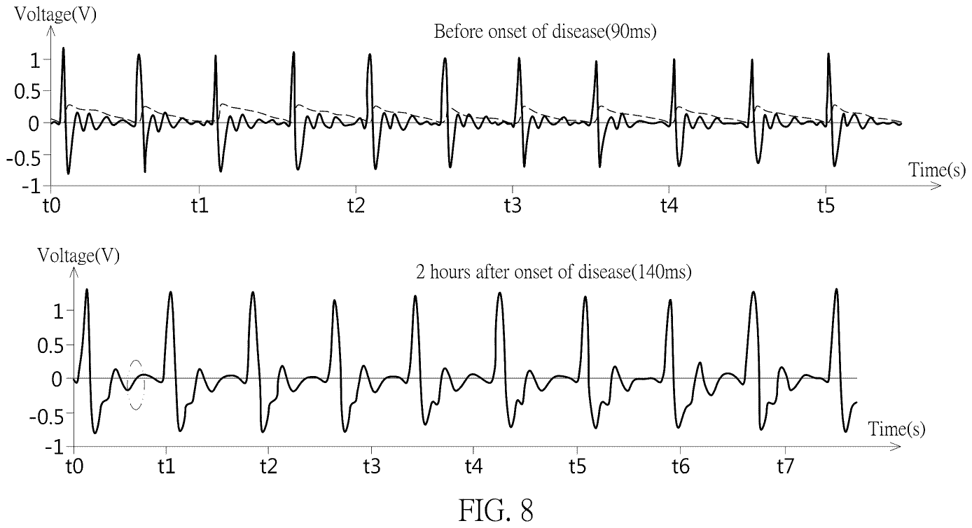

Next, referring to FIG. 8, FIG. 8 shows the characteristic waveforms of patients with paroxysmal palpitations before and during the onset of the disease. It can be seen from the results of each of the characteristic waveforms in FIG. 8 that the characteristic waveform analysis result at the time of onset is that the time interval between the sixth extreme value and the seventh extreme value in a certain heartbeat cycle is detected to be 140 ms, exceeding the preset threshold (110 ms), and the characteristic waveform analysis results before the onset of the disease do not indicate that the time interval between the sixth extreme value and the seventh extreme value exceeds the preset threshold of 110 ms, for example, the maximum is 90 ms. In this way, through the detection, whether or not subjects with diseases related to coronary slow flow are having the onset of the disease can be effectively understood.

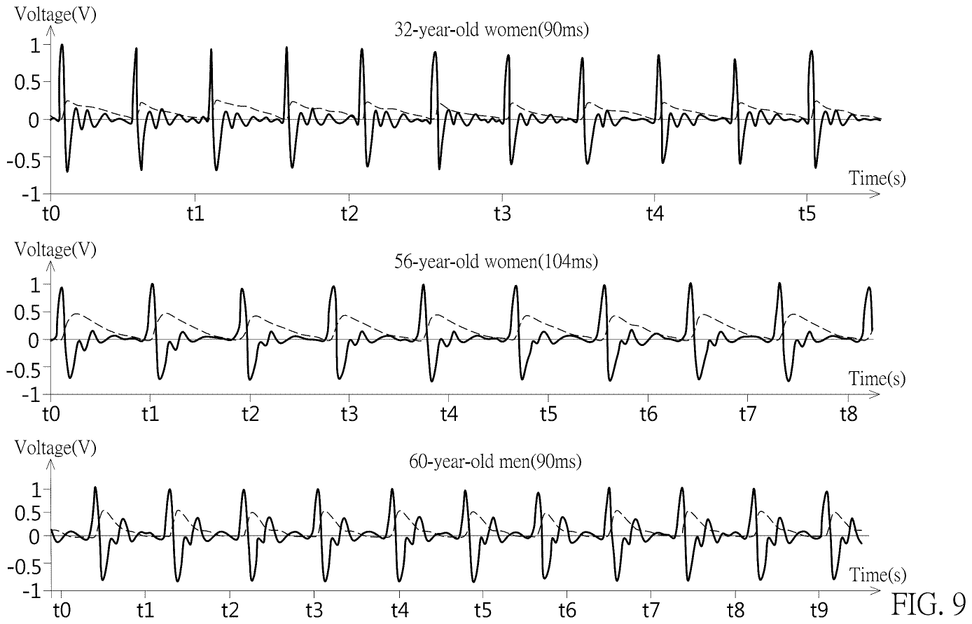

Next, referring to FIG. 9, FIG. 9 shows characteristic waveforms of subjects of different ages and genders. From the marked results of each characteristic waveform in FIG. 9, it can be seen that different ages and genders have little impact on the time interval between the sixth extreme value and the seventh extreme value. For example, in the characteristic waveforms of 32-year-old women, 56-year-old women, and 60-year-old men that are not diagnosed to have symptoms related to coronary slow flow, the maximum time intervals between the sixth extreme value and the seventh extreme value are 90 ms, 104 ms, and 90 ms, all of which are less than the preset threshold of 110 ms.

It should be noted that, in addition to when the time interval between the sixth extreme value and the seventh extreme value is less than a preset threshold (for example, 60 ms), other abnormal phenomena with diagnostic significance may also be represented, such as hypertensive heart diseases related to heart murmur turbulence disturbances, including heart murmurs, turbulent disturbances, and hypertensive heart diseases. Therefore, the marking member 30 can also be used to determine whether or not the time interval between the sixth extreme value and the seventh extreme value of each cycle of the characteristic waveform is less than a preset threshold, and in response to the time interval being less than the preset threshold, the marking member 30 performs marking to form marked data. The preset threshold is preferably 60 ms.

Based on the analysis of examples from FIG. 6 to FIG. 9, in the non-invasive coronary slow flow monitoring devices 100 and 200 of the present disclosure, by detecting the time interval between the sixth extreme value and the seventh extreme value in the characteristic waveform, physiological abnormalities related to coronary slow flow can be effectively identified. The accuracy and versatility of the device in capturing arterial blood flow abnormalities and assessing cardiovascular health can be shown in the changes before and after patients with arrhythmia taking medicine, the difference in waveforms at different time periods in patients with morning hypertension, the comparison before and after the onset of paroxysmal palpitations, and the analysis results of subjects of different ages and genders. In addition, the marked data generated by the non-invasive coronary slow flow monitoring device of the present disclosure can provide diagnostic data to doctors or testing personnel by accumulating the number of abnormal detections or time intervals, further assisting clinical decision-making and evaluating health status of the patient, thereby fully reflecting the special value and practicability of the present disclosure in medical applications.

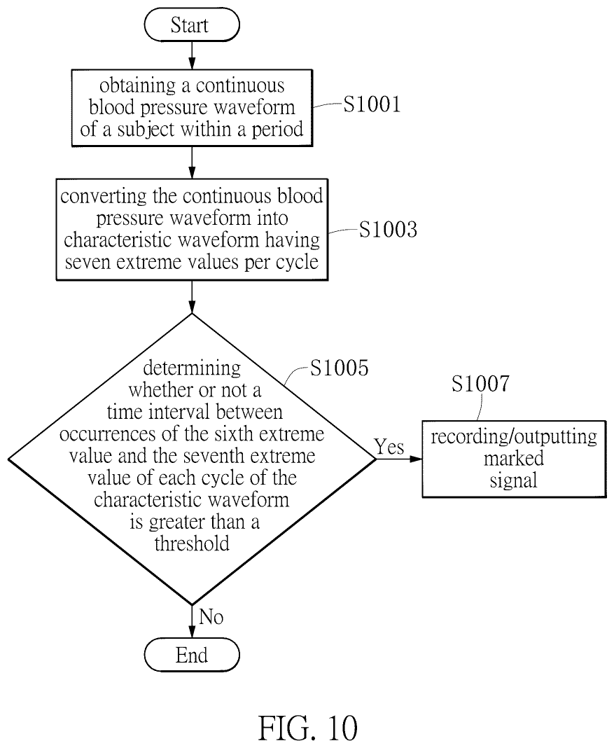

FIG. 10 is a flowchart of a non-invasive coronary slow flow monitoring method according to one embodiment of the present disclosure. The method is run on a control device (such as the control element 1 in FIG. 2) and includes the following steps.

In an acquisition step (S101), the control device obtains at least one continuous blood pressure waveform of a subject within at least one target period.

In an analysis step (S102), the control device converts the continuous blood pressure waveform into a characteristic waveform having at least a first extreme value, a second extreme value, a third extreme value, a fourth extreme value, a fifth extreme value, a sixth extreme value, and a seventh extreme value per cycle.

In a marking step (S103), the control device determines whether or not a time interval between occurrences of the sixth extreme value and the seventh extreme value of each cycle of the characteristic waveform is greater than a preset threshold; in response to the time interval being greater than the preset threshold, the control device outputs a marked signal, or performs marking to form marked data.

In the acquisition step, the control device may control the acquisition member (such as an access device having a wireless/wired transmission function) or an external sensor to obtain at least one continuous blood pressure waveform of the subject within at least one target period. In the analysis step, the control device can process the continuous blood pressure waveform obtained in the acquisition step, and output a characteristic waveform having the first to seventh extreme values P1 to P7 in one cycle. The continuous blood pressure waveform can be a variety of waveforms, including the waveform that reflects the original arterial blood pressure wave, the waveform (the velocity waveform) that reflects the first-order derivative of the original arterial blood pressure wave, or the waveform (the acceleration waveform) that reflects the second-order derivative of the original arterial blood pressure wave. In the marking step, the preset threshold can be from 70 ms to 120 ms according to requirements, for example, the preset threshold can be 70 ms, 80 ms, 90 ms, 100 ms, 110 ms, 120 ms, etc., and preferably is 110 ms. The format and layout of the marked data are not limited herein, and can be referred to in the format of FIG. 3.

In addition, another embodiment of the present disclosure further provides a non-invasive coronary slow flow monitoring device that can efficiently provide physiological data with diagnostic significance and related to coronary slow flow. The difference between this embodiment and the above-mentioned embodiments is that, the marking member can also selectively determine whether or not the time interval between the first extreme value and the second extreme value of each cycle of the characteristic waveform is greater than another preset threshold. In response to the time interval being greater than the another preset threshold, another marked signal is transmitted, or the marking member performs marking to form another marked data. In detail, the marking member may include a determination logic module, a threshold comparison module, and a data recording module. The determination logic module performs analysis according to the characteristic waveform received from the analysis member 20, determines the occurrence time of the first extreme value P1 and the second extreme value P2 in each cycle, and calculates the time interval between the two extreme values. The threshold comparison module calculates the time interval between the first extreme value P1 and the second extreme value P2, and then compares the time interval with the another preset threshold; in response to the time interval being greater than the another preset threshold, the threshold comparison module triggers another marked signal to be output. When the another marked signal is triggered to be output, the data recording module can perform marking and record relevant marked information (such as a subject number, a data reception date and time, a data analysis period, a marking time, a time interval, and an accumulated marking number in the data analysis period, or a combination thereof) to form another marked data.

In addition, other embodiments have described that, when the time interval between the sixth extreme value and the seventh extreme value is less than a preset threshold (for example, 60 ms), other abnormal phenomena with diagnostic significance may also be represented, such hypertensive heart diseases related to heart murmur turbulence disturbances. Therefore, the marking step may include that the control device determines whether or not the time interval between the occurrences of the sixth extreme value and the seventh extreme value of each cycle of the characteristic waveform is less than a preset threshold, and in response to the time interval being less than the preset threshold, the control device outputs the marked signal, or performs marking to form marked data. The preset threshold is preferably 60 ms.

In addition, another embodiment of the present disclosure further provides a non-invasive coronary slow flow monitoring method run on a control device (for example, the control element 1) and includes the following steps. In an acquisition step, the control device obtains at least one continuous blood pressure waveform of a subject within at least one target period. In an analysis step, the control device converts the continuous blood pressure waveform into a characteristic waveform having at least seven extreme values per cycle. In the marking step, the control device determines whether or not a time interval between occurrences of the first extreme value and the second extreme value of each cycle of the characteristic waveform is greater than another preset threshold, and in response to the time interval being greater than the another preset threshold, the control device outputs another marked signal, or performs marking to form another marked data.

A reflected pulse pressure indicates that when blood is pumped from the heart into the arteries, part of the pressure wave is reflected back at arterial branches or places having greater resistances. The reflected pulse pressure will affect the load on the heart, especially when the reflected wave overlaps with the aortic wave, which further increases the load on the heart during the blood pumping process. When the reflected wave returns to the heart, the reflected wave is superimposed on an event wave (i.e., the original pulse wave emanating from the heart), thus causing an increase in central pulse pressure, and simultaneously increasing systolic blood pressure (SBP) and left ventricular afterload (LV afterload). In other words, the heart needs to overcome greater pressure to pump blood out, which can increase the workload on the heart. On the other hand, backflow of the reflected waves can also lead to a decrease in diastolic blood pressure (DBP), which can have a detrimental effect on coronary perfusion (i.e., a blood supply of the heart). When the diastolic blood pressure drops, the heart receives less blood flow during diastole, which affects the self-repair and supply processes of the heart, further exacerbating the risk of heart failure. The reflected pulse pressure can cause the event wave (an event pulse pressure) to rise, especially in cases of high blood pressure or poor heart function. The reflected pulse pressure not only increases the afterload of the heart, causing the heart to use more force to pump blood, but also reduces a coronary blood flow during diastole, thus affecting the health and function of the heart and causing congestive heart failure.

By analyzing whether or not the time interval between the first extreme value P1 and the second extreme value P2 of each cycle exceeds the another preset threshold, the increase in the event pulse pressure caused by the reflected pulse pressure can be effectively detected. Further, through the above-mentioned non-invasive coronary slow flow monitoring device and method of this embodiment, physiological data with diagnostic significance related to congestive heart failure can be efficiently provided.

### Beneficial Effects of the Embodiments

One of the beneficial effects of the present disclosure is that, the non-invasive coronary slow flow monitoring device and method provided by the present disclosure can mark possible abnormalities of the coronary slow flow phenomenon through continuous blood pressure waveforms. A specific subject can be monitored and observed for a long time, and the overall structure is simple and can be placed in a wearable device for daily use and monitoring by the subject. In addition, it can also reduce the cost of obtaining diagnostic data related to the coronary slow flow phenomenon.

Furthermore, with the non-invasive coronary slow flow monitoring device and method provided by the present disclosure, physiological data related to the coronary slow flow and having significance for making medical diagnosis can be obtained without the patient going to a medical institution of a certain scale and undergoing tests with more professional and expensive medical instruments. Furthermore, the subject can be monitored continuously and daily through the non-invasive coronary slow flow monitoring device disposed in the wearable device. Moreover, through the design of the marked data, comparatively more complete physiological data related to the coronary slow flow and having significance for making medical diagnosis can be formed by marking and classifying measurement data according to the condition and time of the subject during the test.

In addition, another non-invasive coronary slow flow monitoring device and method of the present disclosure can analyze whether or not the time interval between the first extreme value P1 and the second extreme value P2 of each cycle exceeds the another preset threshold, so as to further efficiently provide physiological data with diagnostic significance related to congestive heart failure.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A non-invasive coronary slow flow monitoring device, comprising:
an acquisition member used to acquire at least one continuous blood pressure waveform of a subject within at least one target period;
an analysis member communicatively connected to the acquisition member, wherein the analysis member converts the continuous blood pressure waveform acquired by the acquisition member into a characteristic waveform having at least a first extreme value, a second extreme value, a third extreme value, a fourth extreme value, a fifth extreme value, a sixth extreme value, and a seventh extreme value per cycle; and
a marking member communicatively connected to the analysis member, wherein the marking member determines whether or not a time interval between occurrences of the sixth extreme value and the seventh extreme value of each cycle of the characteristic waveform is greater than a preset threshold, and wherein, in response to the time interval being greater than the preset threshold, the marking member outputs a marked signal, or performs marking to form marked data.

2. A non-invasive coronary slow flow monitoring device, comprising:
a control element, including:
an acquisition member used to acquire at least one continuous blood pressure waveform of a subject within at least one target period;
an analysis member used to convert the continuous blood pressure waveform obtained by the acquisition member into a characteristic waveform having at least a first extreme value, a second extreme value, a third extreme value, a fourth extreme value, a fifth extreme value, a sixth extreme value, and a seventh extreme value per cycle; and
a marking member used to determine whether or not a time interval between occurrences of the sixth extreme value and the seventh extreme value of each cycle of the characteristic waveform is greater than a preset threshold, wherein, in response to the time interval being greater than the preset threshold, the marking member performs marking to form marked data;
a memory element communicatively connected to the control element for storing the continuous blood pressure waveform or the marked data; and
a communication element communicatively connected to the control element and the memory element, wherein the communication element is used to externally receive the continuous blood pressure waveform or externally transmit the marked data.

3. The non-invasive coronary slow flow monitoring device according to claim 2, further comprising a sensing element for sensing the subject to obtain the continuous blood pressure waveform.

4. The non-invasive coronary slow flow monitoring device according to claim 2, wherein the marked data includes one or a combination of a subject number, a data reception date and time, a data analysis period, a marking time, a time interval, and an accumulated marking number in the data analysis period.

5. The non-invasive coronary slow flow monitoring device according to claim 1, wherein the continuous blood pressure waveform is the characteristic waveform having at least seven extreme values per cycle.

6. The non-invasive coronary slow flow monitoring device according to claim 1, wherein the preset threshold is 110 ms.

7. The non-invasive coronary slow flow monitoring device according to claim 1, wherein the marking member further determines whether or not a time interval between occurrences of the first extreme value and the second extreme value of each cycle of the characteristic waveform is greater than another preset threshold, and in response to the time interval between the occurrences of the first extreme value and the second extreme value being greater than the another preset threshold, the marking member outputs another marked signal, or performs marking to form another marked data.

8. A non-invasive coronary slow flow monitoring method, running on a control device, and the non-invasive coronary slow flow monitoring method comprising:
an acquisition process, wherein the control device obtains at least one continuous blood pressure waveform of a subject within at least one target period;
an analysis process, wherein the control device converts the continuous blood pressure waveform into a characteristic waveform having at least a first extreme value, a second extreme value, a third extreme value, a fourth extreme value, a fifth extreme value, a sixth extreme value, and a seventh extreme value per cycle; and
a marking process, wherein the control device determines whether or not a time interval between occurrences of the sixth extreme value and the seventh extreme value of each cycle of the characteristic waveform is greater than a preset threshold, and wherein, in response to the time interval being greater than the preset threshold, the control device outputs a marked signal, or performs marking to form marked data.

9. The non-invasive coronary slow flow monitoring method according to claim 8, wherein the preset threshold is 110 ms.

10. The non-invasive coronary slow flow monitoring method according to claim 8, wherein the marking process further includes the control device determining whether or not a time interval between occurrences of the first extreme value and the second extreme value of each cycle of the characteristic waveform is greater than another preset threshold, and in response to the time interval between the occurrences of the first extreme value and the second extreme value being greater than the another preset threshold, the control device outputting another marked signal, or performing marking to form another marked data.
